(19) United States
(12) Patent Application Publication
(10) Pub. No.: US 2026/0355148 A1
(43) Pub. Date: Nov. 12, 2026

(54) PREPARATION METHOD OF FLUOROMETHYL ETHERS AS ACTIVE HYPOGLYCEMIC AGENTS

(71) Applicants: China Three Gorges University, Yichang (CN); Hubei Ruiteli Pharmaceutical Co., Ltd, Yichang (CN)

(72) Inventors: Nianyu HUANG, Yichang (CN); Kaixiong DU, Yichang (CN); Shimei LI, Yichang (CN); Hui YAO, Yichang (CN); Nengzhong WANG, Yichang (CN); Mingguo LIU, Yichang (CN); Linxuan LI, Yichang (CN); Dandan FENG, Yichang (CN)

(21) Appl. No.: 19/697,651

(22) Filed: Jun. 4, 2026

(30) Foreign Application Priority Data
Jun. 9, 2025 (CN) 202510762570.2

(51) Int. Cl. A61K 31/37; A61K 31/121; A61K 31/352; C07C 49/255; C07D 311/16; C07D 311/30

(52) U.S. Cl. CPC A61K 31/37; A61K 31/121; A61K 31/352; C07C 49/255; C07D 311/16; C07D 311/30

(57) ABSTRACT

The present disclosure provides a fluoromethylated natural phenolic compound. A method for preparing fluoromethylated natural phenolic compound is using phenolic natural product and fluoromethyl p-toluenesulfonate as starting materials, potassium carbonate as base, acetonitrile as solvent, followed by reflux to produce the fluorine methylated compound. The reaction yields a single product and is easily isolated, the reaction conditions are mild with good functional group tolerance. The product obtained from the present disclosure has been used in antidiabetic medications, yielding significant results.

### TECHNICAL FIELD

The present disclosure relates to a fluoromethylated natural phenolic compound, especially belongs to the field of pharmaceutical technology.

### BACKGROUND

Over the past few decades, the development of effective methods for introducing fluorine atoms or fluoroalkyl groups into organic molecules has attracted increasing attention in both academia and pharmaceutical industry. In particular, compounds bearing fluoroalkyl groups have emerged as important potential targets in the fields of pharmaceuticals and agrochemicals and have been a focus of recent research, primarily due to high bond energy of C—F bond (485 KJ/mol), which can significantly improve the pharmacokinetic properties of agent molecules. More specifically, as one of strategic fluorinated substituents, monofluoromethyl is widely used in the pharmaceutical industry for many agents and agent candidates, including muscle relaxant fluoroquinolone, anesthetic sevoflurane, anti-inflammatory and anti-asthmatic fluticasone propionate, and hypoglycemic clinical trial agent cameletin.

Among natural bioactive compounds, various components have demonstrated significant anti-diabetic activity, such as polysaccharides, steroids, alkaloids, flavonoids, chalcones, and peptides. These anti-diabetic substances derived from natural sources represent potential resources for the research and treatment of type-2 diabetes. Flavonoids, a class of natural compounds, can regulate glucose absorption and homeostasis, with disulfide ligase as target. Chalcones are precursors of flavonoids and isoflavonoids, widely distributed in plants; many natural chalcones and their derivatives exhibit significant anti-diabetic activity. However, application of natural products is often limited by structural defects inherent to the compounds themselves. Fluoromethyl groups generally possess strong electron-withdrawing properties, lipophilicity, and stable C—F bonds; introducing them into agent molecules can prolong duration of action in vivo and enhance metabolic stability. Furthermore, fluoromethyl groups can increase lipophilicity of agent molecules and their binding affinity to target proteins, thereby improving agent bioavailability. Modifying natural products with hypoglycemic activity using fluoromethyl groups can alter hypoglycemic activity through the characteristics of the fluoromethyl groups. As a 2010 issue of the *Journal of Bioorganic and Medicinal Chemistry* (Mattei, P.; Boehringer, M.; Di Giorgio, P. et al. Discovery of carmegliptin: a potent and long-acting dipeptidyl peptidase IV inhibitor for the treatment of type 2 diabetes [J]. Bioorg. Med. Chem. Lett. 2010, 20 (3), 1109-1113.) reported that cameletin is an effective dipeptidyl peptidase IV inhibitor for the treatment of type-2 diabetes and is currently in the clinical trial phase. The objective of this patent is to establish a synthetic route characterized by readily available starting materials, simple operations, mild conditions, a short process, and high yield. This approach aims to investigate the effects of structural modifications on the α-glucosidase inhibitory activity of various natural bioactive compounds, screen for potential α-glucosidase inhibitors, and ultimately provide a class of natural small-molecule α-glucosidase inhibitors.

### SUMMARY

The present disclosure relates to a fluoromethylated natural phenolic compound, a structural formula of which is represented as:

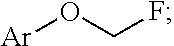

where Ar includes: any of flavonoids, flavonols, chalcone, hydroxychalcone, dihydrochalcone, estrone, sesamol, vanillin, euparin.

In some embodiments, the flavonoids includes: any of chrysin, apigenin, luteolin; the flavanols includes: quercetin or kaempferol; the dihydrochalcone includes: phloretin or hydroxyphloretin.

A preparation method of fluoromethylated natural phenolic compound including:
using phenolic natural product and fluoromethyl p-toluenesulfonate as starting materials, potassium carbonate as base, acetonitrile as solvent, a fluoromethylated a natural phenolic compound is obtained through reflux reaction under nitrogen protection; where a reaction formula is:

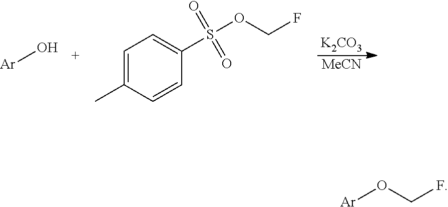

In some embodiments, a molar ratio of natural phenolic product to fluoromethyl p-toluenesulfonate is 2:1.5-3.

In some embodiments, the reflux reaction is conducted at 80-100 °C for 3-5 hours.

Structural formulas of the obtained fluorine methylated compound includes:

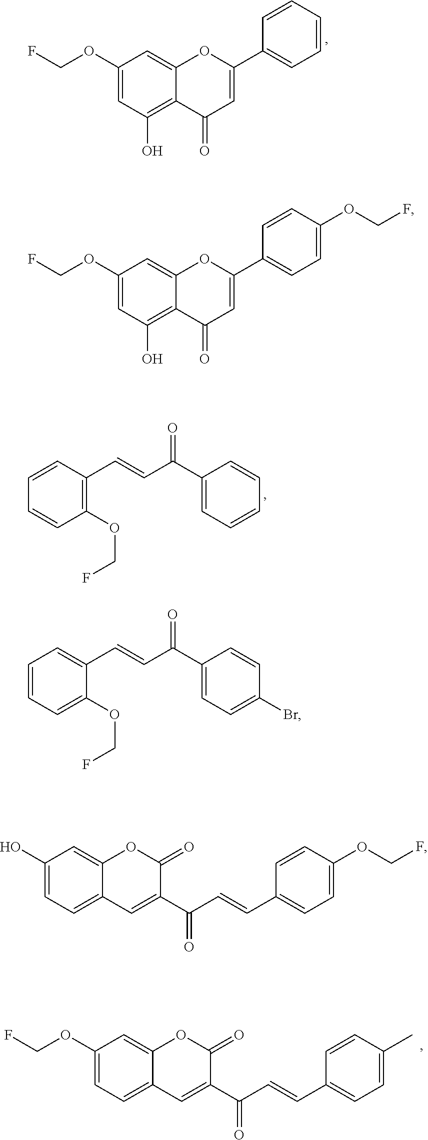 [SMILES: O=c1cc(-c2ccccc2)oc2cc(OCF)cc(O)c12 ; O=c1cc(-c2ccc(OCF)cc2)oc2cc(OCF)cc(O)c12 ; O=C(/C=C/c1ccccc1OCF)c1ccccc1 ; O=C(/C=C/c1ccccc1OCF)c1ccc(Br)cc1 ; O=C(/C=C/c1ccc(OCF)cc1)c1cc2ccc(O)cc2oc1=O ; Cc1ccc(/C=C/C(=O)c2cc3ccc(OCF)cc3oc2=O)cc1]

The present disclosure also provides an application method of the fluoromethylated natural phenolic compound in preparing medications for a treatment of diabetes.

In some embodiments, the application method of the fluoromethylated natural phenolic compound includes an application method in inhibiting α-glucosidase activity for a development of diabetes medications.

The present disclosure also provides a medication for treating diabetes, where the medication including the fluoromethylated natural phenolic compound.

The skilled in the art will appreciate that the described medications may also include pharmaceutically acceptable salts.

The product of the present disclosure is single and easy to separate, and the reaction conditions are mild. The present synthetic method ensures stability of functional groups such as ester groups, ketone carbonyl groups, double bonds, and ether groups, demonstrating excellent functional group tolerance. The products obtained through the present disclosure is used in antidiabetic medications, and achieves significant results.

### BRIEF DESCRIPTION OF THE DRAWINGS

FIG. 1 shows a hydrogen spectrum of compound 3a.

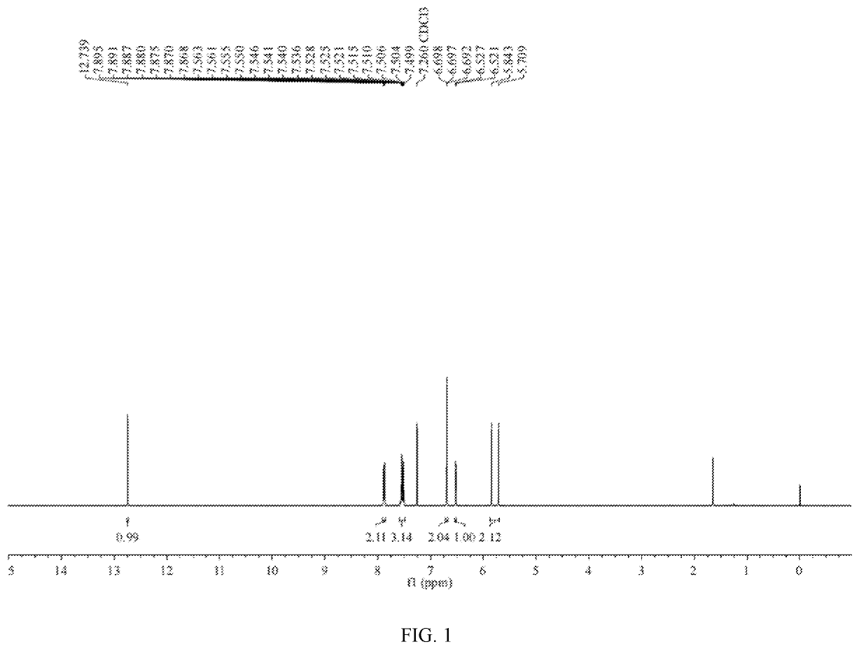

FIG. 2 shows a carbon spectrum of compound 3a.

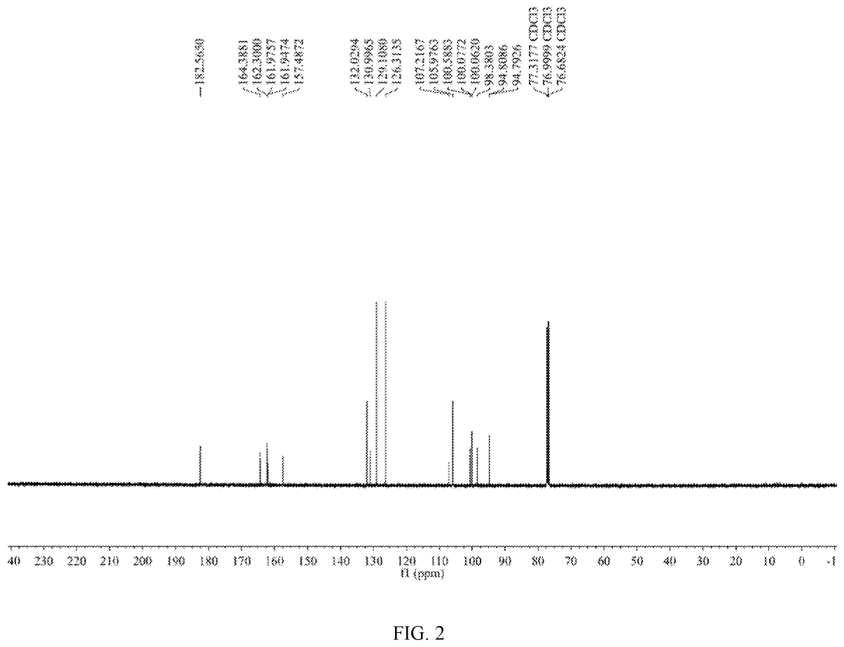

FIG. 3 shows a mass spectrum of compound 3a.

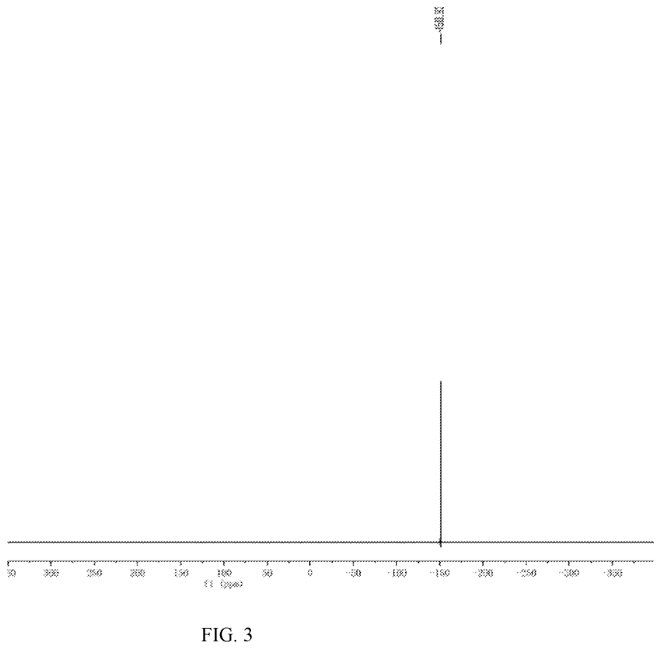

### DETAILED DESCRIPTION

### Experiment Reagent

chrysin (Shanghai McLean Biochemical Technology Co., Ltd), apigenin (Shanghai McLean Biochemical Technology Co., Ltd), sesamol (Shanghai McLean Biochemical Technology Co., Ltd), estrone (Shanghai McLean Biochemical Technology Co., Ltd), 4-Methylumbelliferone (Shanghai McLean Biochemical Technology Co., Ltd), coumarin (Shanghai McLean Biochemical Technology Co., Ltd), potassium carbonate (China National Pharmaceutical Group Chemical Reagent Co., Ltd), acetonitrile (analytically pure, China National Pharmaceutical Group Chemical Reagent Co., Ltd), dichloromethane (analytically pure, Tianjin Kemio Chemical Reagent Co., Ltd), petroleum ether (boiling range 60-90 °C, Tianjin Hengxing Chemical Reagent Manufacturing Co., Ltd), ethyl acetate (analytically pure, Tianjin Kemio Chemical Reagent Co., Ltd), anhydrous sodium sulfate (analytically pure, China National Pharmaceutical Group Chemical Reagent Co., Ltd), deuterated chloroform (deuterium 99.8%, TMS 0.03% V/V, 10*0.5 mL/box, ARMAR, Switzerland), NMR tube (5 mm 100/pk 2 ST500-8, Norell, Inc. (U.S.)).

### Experimental Apparatus

ZXZ-4 rotary vane vacuum pump (Linhai Tanshi Vacuum Equipment Co., Ltd), DZF-6020 vacuum drying oven (Shanghai Xinmiao Medical Equipment Manufacturing Co., Ltd), SHB-IIIA circulating water multi-purpose vacuum pump (Shanghai Yukang Science and Education Instrument Equipment Co., Ltd), CL-4 plate magnetic stirrer (Zhengzhou Changcheng Technology Industry and Trade Co., Ltd), EYELA SB-1100 rotating evaporator (Shanghai Ailang Instrument Co., Ltd), FA2104B analytical balance (Shanghai Yueping Technology Instrument Co., Ltd), XRC-1 micro-melting point apparatus (Sichuan University Science and Instrument Factory), DF-101S heat-collecting constant temperature heating magnetic stirrer (Gongyi Yingyu Yuhua Instrument Factory), ZF-6 three-purpose ultraviolet analyzer (Shanghai Jiapeng Technology Co., Ltd), Ultrashied 400 MHz Plus nuclear magnetic resonance apparatus (Bruker Switzerland).

### Embodiment 1

use natural phenolic products and fluoromethyl p-toluenesulfonate as raw materials, potassium carbonate as a base, and acetonitrile as a solvent to conduct a reflux reaction at 80 °C, details are:

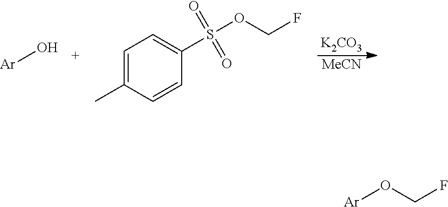

technical route is:
add natural phenolic products 1a~1f (0.2 mmol, 1.0 equiv.) to a 5 mL reaction tube, then add 4.0 mL acetonitrile as solvent, adds potassium carbonate (2.0 mmol, 10.0 equiv.), and finally add fluoromethyl p-toluenesulfonate (0.3 mmol, 1.5 equiv.). Carry out the reaction at 80 °C for 4 h and detected by TLC in time. After the reaction is completed, add water to quench the reaction. Extract the reaction solution with ethyl acetate, separate and repeat three times. Collect organic phase and dry with anhydrous sodium sulfate. Obtain crude product after concentration by rotary evaporator. Let the crude product go through column chromatography separation, where a eluent system PE:EA=10:1. After separation, vacuum-dried product to yield fluoromethylated products 3a-3f, with yields of 87.3% to 93.2%. There are some variations in yields of products corresponding to different synthons, which may be related to factors such as structural characteristics and reactivity of synthons themselves.

### Substrate Range

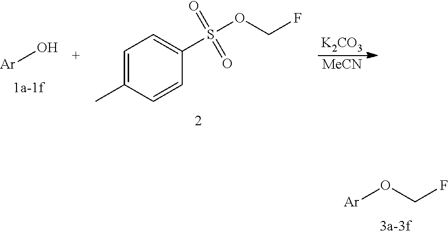

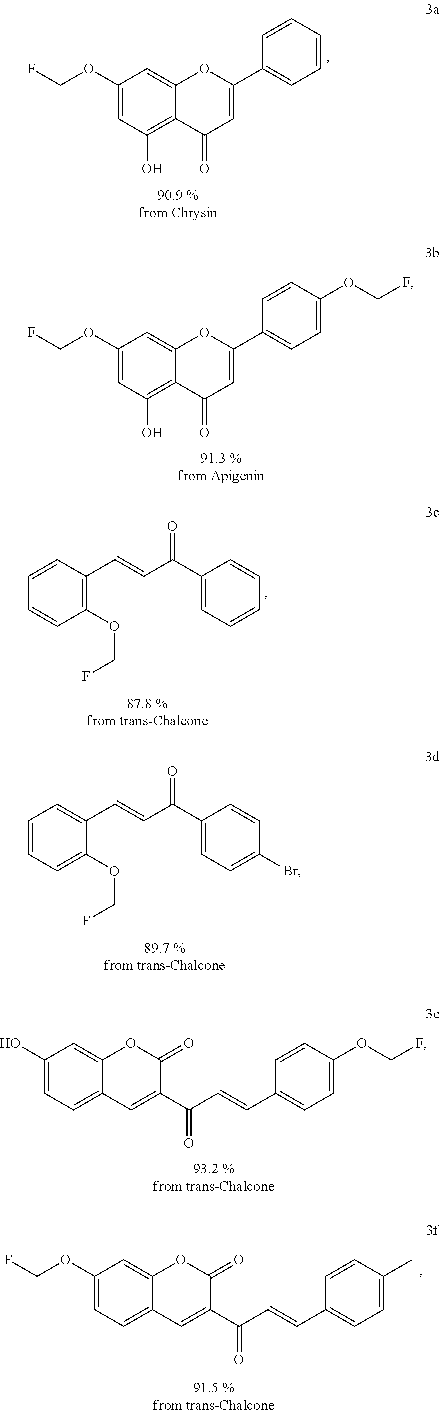 [SMILES: O=c1cc(-c2ccccc2)oc2cc(OCF)cc(O)c12 ; O=c1cc(-c2ccc(OCF)cc2)oc2cc(OCF)cc(O)c12 ; O=C(/C=C/c1ccccc1OCF)c1ccccc1 ; O=C(/C=C/c1ccccc1OCF)c1ccc(Br)cc1 ; O=C(/C=C/c1ccc(OCF)cc1)c1cc2ccc(O)cc2oc1=O ; Cc1ccc(/C=C/C(=O)c2cc3ccc(OCF)cc3oc2=O)cc1]

Corresponding 1a-1f is reaction raw material of the corresponding 3a-3f, and a terminal is a hydroxyl structure.

### Spectra Data

### 7-(fluoromethoxy)-5-hydroxy-2-phenyl-4H-chromen-4-one (3a)

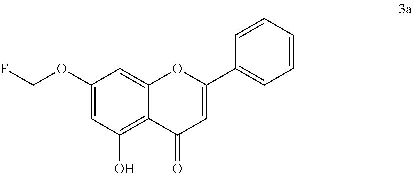 [SMILES: O=c1cc(-c2ccccc2)oc2cc(OCF)cc(O)c12]

¹H NMR (400 MHZ, CDCl₃) δ 12.74 (s, 1H), 7.90-7.87 (m, 2H), 7.56-7.50 (m, 3H), 6.70-6.69 (m, 2H), 6.52 (d, J=2.4 Hz, 1H), 5.78 (d, J=53.5 Hz, 2H). ¹³C {¹H} NMR (100 MHZ, CDCl₃) δ 182.6, 164.4, 162.3, 162.0 (J=2.8 Hz), 157.5, 132.0, 131.0, 129.1, 126.3, 107.2, 106.0, 100.1, 99.5 (J=220.5 Hz), 94.8. ¹⁹F NMR (376 MHz, CDCl₃) δ −150.94. HRMS (ESI) m/z: calcd. for C₁₆H₁₁FO₄Na⁺ (M+Na)⁺, 309.0539, found 309.0534.

### Embodiment 2

use natural phenolic products and methyl iodomethane as raw materials, potassium carbonate as a base, and acetonitrile as a solvent to conduct a reflux reaction at 40 °C, details are:

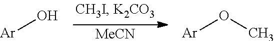

technical route is:
add natural phenolic products 1a~1f (0.2 mmol, 1.0 equiv.) to a 5 mL reaction tube, then add 4.0 mL acetonitrile as solvent, adds potassium carbonate (2.0 mmol, 10.0 equiv.), and finally add methyl iodomethane (0.3 mmol, 1.5 equiv.). Carry out the reaction at 40 °C for 4 h and detected by TLC in time. After the reaction is completed, add water to quench the reaction. Extract the reaction solution with ethyl acetate, separate and repeat three times. Collect organic phase and dry with anhydrous sodium sulfate. Obtain crude product after concentration by rotary evaporator. Let the crude product go through column chromatography separation, where a eluent system PE:EA=10:1. After separation, vacuum-dried product to yield methylate products 4a-4f, with yields of 75% to 84%.

### Substrate Range

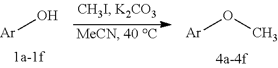

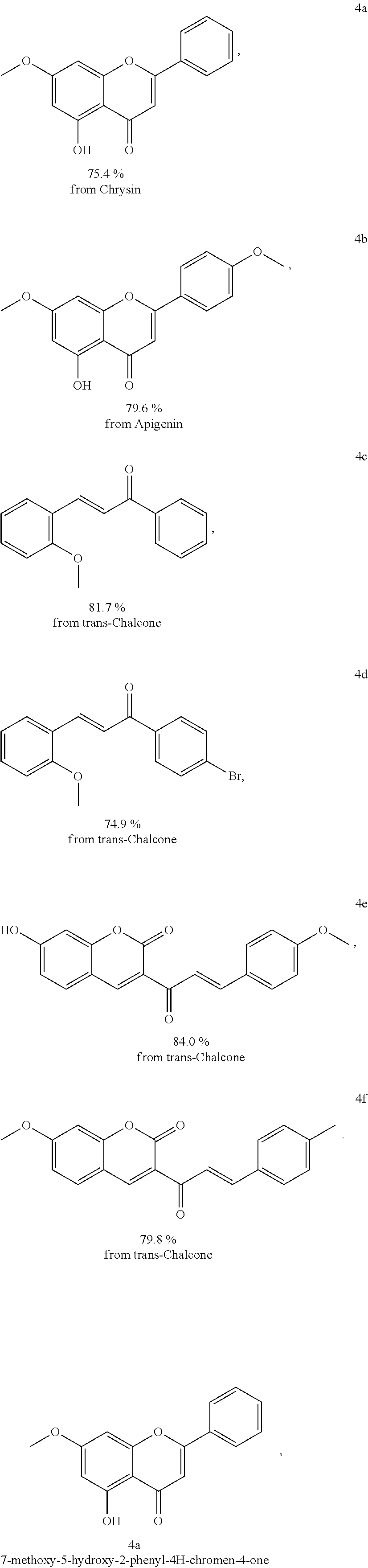

¹H NMR (400 MHz, CDCl₃) δ 12.76 (s, 1H), 7.92-7.85 (m, 2H), 7.58-7.52 (m, 3H), 6.68 (s, 1H), 6.50 (s, 1H), 6.39 (s, 1H), 3.89 (s, 3H). HRMS (ESI) m/z: calcd, for C₁₆H₁₂O₄Na⁺ (M+Na)⁺, 291.0633, found 291.0636.

### Embodiment 3

Evaluation of α-glucosidase inhibitory activity of fluoromethyl ether active molecules:
prepare reaction solution: PBS solution (0.1 mM, pH 6.8), substrate PNPG solution (4 mM), α-glucosidase solution (0.2 U/mL), Na₂CO₃ solution (0.2 mol/L).

The experiment is divided into enzyme activity group (50 μL α-glucosidase solution and 20 μL PBS solution), enzyme blank group (70 μL PBS solution), sampling group (50 μL α-glucosidase solution and 20 μL sample solution), positive group (50 μL α-glucosidase solution and 20 μL positive medication solution), sample blank group (50 μL PBS solution and 20 μL sample solution), positive blank group (50 μL PBS solution and 20 μL positive medication solution). Each group of medications is added to a 96-well plate according to the above dose, three parallel in each group, shake and mix for 2 min, react at 37 °C for 10 min, add 25 μL PNPG solution, shake and mix for 2 min, react at 37 °C for 30 min, add 100 μL Na₂CO₃ solution to terminate the reaction, shake and mix for 5 min. Since PNPG can hydrolyzes into glucose and PNP under an action of α-glucosidase, PNP exhibits maximum absorption at 405 nm, measure the absorbance, and calculate inhibition rates and IC₅₀ values of α-glucosidase for each sample using a calculation formula.

### Calculation Formula:

Inhibitory rate=(1−Δ_{Sample}/Δ_{Enzyme})×100%;
Δₛₐₘₚₗₑ=average OD value of sample-blank OD value of sample;
Δ_{Enzyme}=average OD value of enzyme activity-blank OD value of nzyme activity.

Evaluation results of α-glucosidase inhibitory activity of fluoromethyl ether active molecules

| Compound | IC₅₀ (mmol/L) | Compound | IC₅₀ (mmol/L) | Compound | IC₅₀ (mmol/L) |
|---|---|---|---|---|---|
| 1a | 16.58 ± 0.11 | 3a | 0.35 ± 0.03 | 4a | 85.4 ± 0.15 |
| 1b | 9.83 ± 0.12 | 3b | 0.22 ± 0.02 | 4b | >100 |
| 1c | 27.62 ± 0.31 | 3c | 0.77 ± 0.01 | 4c | >100 |
| 1d | 26.91 ± 0.14 | 3d | 0.65 ± 0.02 | 4d | >100 |
| 1e | 14.25 ± 0.10 | 3e | 0.38 ± 0.03 | 4e | 76.1 ± 0.33 |
| 1f | 30.79 ± 0.11 | 3f | 0.57 ± 0.02 | 4f | >100 |
| Acarbose | 16.10 ± 0.34 | | | | |

What is claimed is:

1. A fluoromethylated natural phenolic compound, a structural formula of which is represented as: 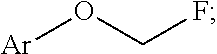
wherein Ar comprises: any of flavonoids, flavonols, chalcone, hydroxychalcone, dihydrochalcone, estrone, sesamol, vanillin, euparin.

2. The fluoromethylated natural phenolic compound according to claim 1, wherein the flavonoids comprises: any of chrysin, apigenin, luteolin; the flavanols comprises: quercetin or kaempferol; the dihydrochalcone comprises: phloretin or hydroxyphloretin.

3. A preparation method of fluoromethylated natural phenolic compound according to claim 1, comprising:
using phenolic natural product and fluoromethyl p-toluenesulfonate as starting materials, potassium carbonate as base, acetonitrile as solvent, a fluoromethylated a natural phenolic compound is obtained through reflux reaction under nitrogen protection.

4. The preparation method of fluoromethylated natural phenolic compound according to claim 3, wherein a molar ratio of natural phenolic product to fluoromethyl p-toluenesulfonate is 2:1.5-3.

5. The preparation method of fluoromethylated natural phenolic compound according to claim 3, wherein the reflux reaction is conducted at 80-100 °C for 3-5 hours.

6. The preparation method of fluoromethylated natural phenolic compound according to claim 3, wherein structural formulas of the obtained fluorine methylated compound comprises:

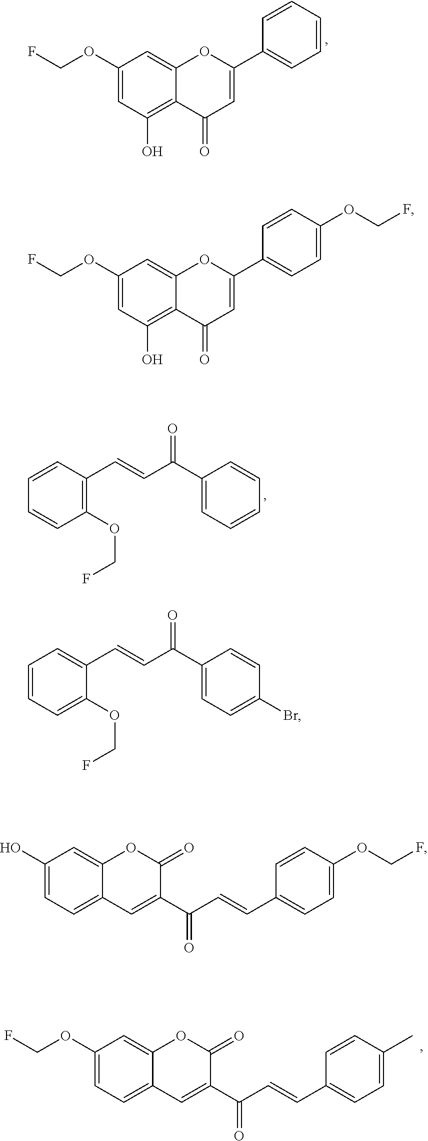 [SMILES: O=c1cc(-c2ccccc2)oc2cc(OCF)cc(O)c12 ; O=c1cc(-c2ccc(OCF)cc2)oc2cc(OCF)cc(O)c12 ; O=C(/C=C/c1ccccc1OCF)c1ccccc1 ; O=C(/C=C/c1ccccc1OCF)c1ccc(Br)cc1 ; O=C(/C=C/c1ccc(OCF)cc1)c1cc2ccc(O)cc2oc1=O ; Cc1ccc(/C=C/C(=O)c2cc3ccc(OCF)cc3oc2=O)cc1]

7. An application method of the fluoromethylated natural phenolic compound according to claim 1 in preparing medications for a treatment of diabetes.

8. The application method of the fluoromethylated natural phenolic compound according to claim 7, wherein the application method of the fluoromethylated natural phenolic compound in inhibiting α-glucosidase activity for a development of diabetes medications.

9. A medication for treating diabetes, wherein the medication comprising the fluoromethylated natural phenolic compound according to claim 1.

10. The medication for treating diabetes according to claim 9, wherein the medication comprises pharmaceutically acceptable salts.
